(19) United States
(12) Patent Application Publication
(10) Pub. No.: US 2023/0170050 A1
(43) Pub. Date: Jun. 1, 2023

(54) SYSTEM AND METHOD FOR PROFILING ANTIBODIES WITH HIGH-CONTENT SCREENING (HCS)

(71) Applicant: PHENOMIC AI, Toronto (CA)

(72) Inventors: Sam COOPER, Toronto (CA); Oren KRAUS, Toronto (CA); Max LONDON, Toronto (CA); Grant WATSON, Toronto (CA); Allison NIXON, Toronto (CA); Elizabeth KOCH, Toronto (CA); Ètienne DUMOULIN, Toronto (CA); Arif JETHA, Toronto (CA)

(21) Appl. No.: 17/918,882

(22) PCT Filed: Apr. 16, 2021
(86) PCT No.: PCT/CA2021/050524

§ 371 Date: Oct. 13, 2022

Related U.S. Application Data
(60) Provisional application No. 63/011,999, filed on Apr. 17, 2020.

(51) Int. Cl. G16B 40/20; G16B 35/20; G06N 3/0895

(52) U.S. Cl. CPC G16B 40/20; G16B 35/20; G06N 3/0895

(57) ABSTRACT

Systems and methods that receive as input microscopy images, extract features, and apply layers of processing units to compute one or more sets of cellular phenotype features, particularly antibodies, corresponding to cellular densities and/or fluorescence measured under different conditions. The system is a machine learning architecture having, in one aspect, a deep neural network, typically a convolutional neural network. The deep neural network can be trained and tested directly on raw microscopy images. The system computes class specific feature maps for every phenotype variable using a deep neural network. The system produces predictions for one or more reference antibody variables based on microscopy images within populations of cells.

### BACKGROUND OF THE INVENTION

### Field of the Invention

The present invention relates to a system and method for grouping antibodies by their phenotypic effect on cells and experimental cell models (Cell-based assays), employing neural networks.

### Description of the Conventional Art

In current antibody drug discovery pipelines, initial focus and triaging is typically placed on antibody binding affinity, and biophysical properties of the antibody such as thermal stability or solubility. Subsequently, antibodies are progressed into simple cell-based models of a signaling pathway (simple cell-based assays), that determine whether they have biological efficacy. For example, a fluorescent reporter of signaling activity may be introduced into a signaling pathway, and the level of signaling activity determined by measuring the fluorescence levels across a population of cells in a multi-well plate. The ability of an antibody to inhibit signaling is then quantified by a one-dimensional readout of pathway inhibition. Other examples of one-dimensional assays commonly used to measure the biological effect of an antibody include measuring target transcript levels by qPCR, quantifying total target protein levels by western blotting, and quantifying the release of cytokines by capture enzyme-linked immunosorbent assay (ELISA).

The set of antibodies which are active against a simple cell-based assay are then grouped by the biophysical location of where they bind their target protein, in a process known as epitope binning. Lead candidates are then typically taken from the major epitope bins for further characterization and in vivo follow up. Yet throughout these early stages of discovery, little emphasis is placed on effects that an antibody might be having on cells outside of the assumed effects of binding a target—as measured with a simple cell-based assay.

This creates a problem since simple cell-based assays typically fail to capture the biological complexity witnessed in human disease, and as a result may lead to the prioritization of antibodies which are ineffective in situ (in human clinical trials). Moreover, the assumption that all antibodies in an epitope group behave similarly is also not true, since antibodies with different affinities and effects on target protein structure can lead to different behaviors. Thus, by basing selection of antibodies for follow-up study on epitope groupings, candidates may be missed that have more desirable effects than those tested. Thus, there is a need in the art for systems and methods for improved high throughput screening of antibodies for desirable therapeutic and/or diagnostic attributes.

### SUMMARY OF THE INVENTION

In accordance with aspects of the present invention, potential therapeutic antibodies are profiled against high-dimensional, image-based phenotypic assays quantified by WSE. With this approach, on-target, off-target, and previously undiscovered biological effects of an antibody can be profiled in a single experimental assay, or through the merger of multiple assays to add further experimental dimensions to the feature vector. The resulting process compresses the number of steps and the amount of effort and time required to accomplish the grouping of antibodies by their phenotypic effect, termed ‘phenotype binning’. The method also allows the application of phenotypic binning to arbitrarily complex cell-based assays. At the same time the process enhances the overall sensitivity, and consistency, of the system employed for detecting the phenotypic effect of antibodies in cell-based assays, by employing many more quantitative features than traditional methods use and providing capability well beyond the capabilities of humans who otherwise would “eyeball” individual images to try to obtain the same results. Taken together, the present invention enables the creation and use of phenotypic bins either alongside, or instead of, epitope bins for guiding the selection of antibodies for follow-up studies in the drug discovery pipeline.

Much as epitope bins can be used to identify key elements of an antibody's structure that drive its binding to an epitope; phenotypic bins can also be used to identify key structural elements that drive a specific phenotypic effect. To build these structure-activity relationships large numbers of antibodies with different sequences must be tested and grouped consistently with quantitative approaches. Thus, it may also be desirable to use quantitatively defined phenotypic groups, as generated by the process described herein, to identify regions of antibody variable heavy (VH) and variable light (VL) domains that correlate with specific phenotypic effects. The establishment of such a structure-phenotypic activity relationship can then be used to guide rational modification of antibody sequences.

In one aspect, the invention provides a method for profiling antibodies comprising: providing a machine learning model receiving one or more inputs taken from an image selected from one of a plurality of images generated from one or more groups of imaging assays, wherein the image is associated with other images of the one or more groups according to one or more antibodies present in a biological sample from which the image was generated; and using the machine learning model, computing an output comprising one or more predicted phenotypes represented in one or more of the plurality of images of that group, the output comprising an antibody profile. In some embodiments, the biological sample comprises a plurality of cells. In some embodiments, the plurality of cells comprises two or more different cell types. In preferred embodiments, the machine learning process is weakly supervised, and the model is a neural network, preferably selected from the group of a multiple instance learning network (MIL) model, a deep neural network (DNN), a convolutional neural network (CNN), and a neural network comprising one or more convolutional layers and an MIL pooling layer.

In some embodiments, the method further comprises training the machine learning model using the plurality of images, to enable the model to predict a group from the one or more groups of which the image is a member; preferably wherein the one or more groups come from a common experimental design with other images. In some embodiments the training further comprises enabling the model to predict whether an image from a group was generated from one or a plurality of experimental conditions in the assays from which the images were generated. In some embodiments, the experimental conditions are selected from the set of antibody identifier, concentration of antibody, cell type, combination of cell types, cell seeding density, probe set, presence, absence or concentration of ligand, presence, absence or concentration of small molecule inhibitors, depletion, knockout, overexpression or modulation of genes, or presence, absence or concentration of combinations of any other molecules that may modulate the activity of an antibody, and the like.

In some embodiments, the output of the method further comprises providing a graphical display of phenotype versus antibody class grouped according to a distribution of learned weights in one or more hidden layers of the trained machine learning model.

In some embodiments, the inputs comprise pixel intensities. In preferred embodiments, the pixel intensities correspond to a plurality of fluorescent probes in said biological sample in said assay, each probe representing a phenotype of interest. Still more preferably, at least one probe represents an on-target phenotype and at least one probe represents an off-target phenotype. In some embodiments, the on-target phenotype is cell stimulation and/or expansion. In some embodiments, the on-target phenotype is cell senescence, apoptosis and/or cytotoxicity. In some embodiments, the on-target phenotype is stimulation of a cell-signaling pathway. In some embodiments, the on-target phenotype is inhibition of a cell-signaling pathway.

In some embodiments, the output comprises a graphical representation of the antibody profile. In some embodiments, the output antibody profile comprises a predicted classification of phenotype, wherein the phenotype is selected from the group consisting of on-target and off-target. In some embodiments, the on-target phenotype comprises inhibition of an activated state of a tumor cell, a non-tumor cell, or a cell in contact with a tumor cell.

In exemplary embodiments, the on-target phenotype comprises inhibition of activation of a non-tumor cell fibroblast by an exogenously applied activating ligand. In exemplary embodiments, the activating ligand is TGF-beta. In some embodiments, the cell in contact with the tumor cell is a fibroblast, and the on-target phenotype comprises inhibition of tumor cell contact-induced fibroblast activation.

In some embodiments, the off-target phenotype is selected from the group consisting of autophagy, cytotoxicity, auto-fluorescence, and senescence induction.

In additional embodiments, the method further comprises a preliminary step of individually contacting each of a panel of antibodies with a biological sample, wherein said sample comprises a probe set representative of a plurality of phenotypes of interest, and generating at least one image of each antibody/biological sample pairing in said panel. In some embodiments, the method comprises generating two or more images of each antibody/biological sample pairing at sequential time points. In some embodiments, at least one probe represents an on-target phenotype and at least one probe represents an off-target phenotype.

In another aspect, the invention provides a method for profiling antibodies based on phenotypic effect and/or activity, comprising the steps of: a) contacting a plurality of antibodies with a biological sample in an arrayed format, wherein said biological sample comprises one or more cell types comprising a plurality of labeled probes to create a high-content assay; b) imaging said high content assay with automated microscopy to generate an imaging dataset; and c) applying a deep neural network to said imaging dataset to detect the set of phenotypes present in the imaging dataset; and determine the antibodies that induce one or more of these phenotypes.

In preferred embodiments, weakly supervised embedding is used to train the deep neural network on the phenotypic similarity between different images. In some embodiments, the deep neural network is trained on a plurality of extracted features encompassing variations between regions of interest in each image in the dataset, and to embed the imaging dataset. In some embodiments, regions of interest comprising extracted features that are unique to an experimental condition are passed into the deep neural network and the result of the prediction on a subset of training data is used to directly update the weights in the deep neural network. In exemplary embodiments, an unsupervised clustering technique is used to identify discrete phenotypic groups defined by a threshold level of similarity between extracted features.

As illustrated in FIG. 1A, representative inputs to the inventive methods provided herein comprise: 1) an experimental assay of a biological system comprising one or more cell types illuminated with a set of fluorescent probes or dyes (e.g., 10 or more, 25 or more, 50 or more, 100 or more) to create a “high-content assay” capturing aspects of the target biology, e.g. activation, alongside other phenotypic behaviors e.g. cytotoxicity, which can be imaged using automated microscopy to generate “high-content” images; and 2) a set of antibodies (e.g., 10 or more, 25 or more, 50 or more, 100 or more) comprising a plurality of VH and VL domains that are hypothesized to have biological activity against the high-content assay.

As noted above, aspects of the invention include a procedure to identify and profile the effects of antibodies on biological systems. First, antibody protein is provided in an arrayed format to the cells seeded as per the high-content assay protocol. In an exemplary embodiment, multiple unique variable heavy and light chain sequences are synthesized as DNA oligomers and are ligated into common heavy and light chain scaffolds, that can be part of the same or independent expression vectors, and each expression vector is then infected or transfected into an expression system to produce antibody protein. Preferably, the infection/transfection is carried out in an arrayed format such that each antibody is produced in isolation. In particularly preferred embodiments, independent vectors are used for heavy and light chains and can be tested combinatorially, such that all heavy and light chains can be tested with one another as a means to generate additional diversity.

Next, after a predetermined period of time has passed, the high content cell-based assay is either fixed and stained, or imaged using live-cell reporters, with automated microscopy to generate an imaging dataset—as per the high-content assay protocol, as per FIG. 1B.

Thirdly, a deep-neural network that is either pre-trained on other experimental datasets, or trained from scratch random weight initializations on the set of images produced from the imaging assays, is then used to detect the set of phenotypes present in the imaging dataset; and the antibodies that induce these phenotypes. Additionally, the deep-neural network can be used to classify phenotypes that are induced by different antibodies against a set of experimental controls. As discussed herein, various types of neural networks may be suited to this technique.

In an exemplary embodiment, WSE is used to learn a distance metric that captures the phenotypic similarity between different images. Specifically, a neural network is trained to predict which experimental condition, or field of view, a region of interest belongs to. In doing so the network learns which, if any, features of that region of interest are unique to an experimental condition. If features exist which are unique to an experimental condition these conditions separate from other conditions in the learnt feature space. If no unique features exist, the learnt feature space is unable to separate the given condition from one or more other conditions. Thus, an effective distance metric describing the similarity between images is learnt.

Where WSE is used to train a multi-layer neural network on extracted features, the first step is to extract a plurality of features from the set of images in the dataset, e.g., 5 or more, 10 or more, 20 or more, 30 or more, 40 or more, 50 or more, etc., preferably wherein the extracted features are designed to encompass most or all variations between images within the dataset. A neural network is then trained on the extracted features to embed the imaging dataset. Where WSE is used to train deep convolutional neural networks (CNN's) and variations on these networks such as deep multiple instance learning networks (MIL), regions of interest are passed into the deep-neural network and the result of the prediction on a subset of training data is used to directly update the weights in the CNN. As such raw imaging data is directly embedded. This is detailed in FIG. 1C.

Finally, one or more phenotypic groups, or bins, are identified in the high-dimensional embedded data. In an exemplary embodiment, this occurs through the application of unsupervised clustering techniques such as K-means clustering and hierarchical clustering to identify discrete groups of effects as defined by a threshold level of similarity; optionally combined with visualization of the high-dimensional embeddings using methods for projecting high-dimensional data into 2D subspaces to verify whether discrete phenotypic groups are consistent with the local and global structure of the embedded dataset; and/or mapping of individual features extracted from imaging data to phenotypic groups to ensure distinct biological differences (e.g. variation in cell size, protein localization, immunofluorescent probe intensity) are driving the separation of phenotypic groups, rather than technical artifacts such as background intensity differences.

The output from the process will be the set of antibody sequences separated into groups that encompass the phenotypic effect of the antibodies on a specific high-content assay.

In one aspect, a subset of representative antibodies from a phenotypic bin may be further investigated in the drug-discovery pipeline—and by not having to progress every antibody into follow-up studies, e.g. in vivo mouse models, testing cost and time is saved. It is also assumed that by selecting representative antibodies from different phenotypic bins a significant percentage of the set of all possible outcomes is characterized in follow-up studies.

In another aspect, directed mutagenesis, or tailored libraries of antibodies may be generated based on the sequence characteristics of a phenotypic group of antibodies; it is hypothesized that a subset of the mutagenesis-derived antibodies will have the same phenotypic effect as those previously identified, although this effect may be seen following application of a lower concentration of antibody, or that the antibodies in this subset will have improved, or ‘more drug-like’, physiochemical properties that are desirable for manufacture and use of the antibody as a therapeutic.

In another aspect, antibodies may be optimized toward an experimental control. For example, ‘healthy phenotype’ mutations can be made to the antibodies that have a phenotypic effect closest to the positive experimental control and the process iterated, leading to directed evolution of antibody sequences towards those that produce the desired phenotype. Where the objective is to optimize for new phenotypes, mutations can be made to those antibodies furthest from the negative control and the process iterated to enhance phenotypic effects versus negative controls.

In a further aspect, the invention provides a computer-implemented machine learning architecture for profiling antibody activity in a high-content assay, the machine learning architecture executed on one or more processing units, the machine learning architecture comprising: a machine learning model receiving one or more inputs taken from an image selected from one of a plurality of images generated from one or more groups of imaging assays, wherein the image is associated with other images of the one or more groups according to one or more experimental conditions including antibody treatments present in a biological sample from which the image was generated; the machine learning model comprising an input layer receiving the one or more inputs, and one or more hidden layers of processing nodes, each processing node comprising a processor configured to apply an activation function and a weight to inputs of the processor, a first of the hidden layers receiving an output of the input layer and each subsequent hidden layer receiving an output of a prior hidden layer; and at least one of the one or more hidden layers configured to generate one or more class specific predictions for cellular features of one or more cell classes present in the images, wherein the class specific predictions represent probabilities of the cell classes for an image; and an output layer, responsive to the learned weights from one or more of the hidden layers and the probabilities of the cell classes, to generate an antibody profile according to the weights and probabilities of the cell classes. In some embodiments, the antibody profile comprises a graphical representation. In some embodiments, one or more of the hidden layers comprise a convolutional layer. In some embodiments, the machine learning model comprises a neural network selected from the group comprising or consisting of an MIL model, a deep neural network (DNN), a convolutional neural network (CNN), and a neural network comprising one or more layers.

In another embodiment, the invention provides a computer-implemented machine learning architecture for profiling antibody activity in a high-content assay, the machine learning architecture executed on one or more processing units, the machine learning architecture comprising: a machine learning model receiving one or more inputs taken from an image selected from one of a plurality of images generated from one or more groups of imaging assays, wherein the image is associated with other images of the one or more groups according to one or more antibodies present in a biological sample from which the image was generated; the machine learning model comprising an input layer receiving the one or more inputs, and one or more hidden layers of processing nodes, each processing node comprising a processor configured to apply an activation function and a weight to inputs of the processor, a first of the hidden layers receiving an output of the input layer and each subsequent hidden layer receiving an output of a prior hidden layer; and at least one of the one or more hidden layers configured to generate one or more class specific predictions for cellular features of one or more cell classes present in the images, wherein the class specific predictions represent probabilities of the cell classes for an image; a global pooling layer configured to receive the one or more class specific predictions for cellular features and to apply a multiple instance learning (MIL) pooling function to combine respective probabilities from each class specific prediction to produce a prediction for each cell class present in the image; and an output layer, responsive to the learned weights from one or more of the hidden layers and the probabilities of the cell classes, to generate an antibody profile according to the weights and probabilities of the cell classes. In some embodiments, the antibody profile comprises a graphical representation. In some embodiments, the machine learning model is trained to identify whether the image is from one of a set of experimental conditions from which the image was generated. In some embodiments, the machine learning model is trained to identify whether the image is predicted to depict a positive result indicating the antibody produces a phenotype corresponding to a positive control image or a negative result indicating the antibody produces a phenotype corresponding to a negative control image.

### BRIEF DESCRIPTION OF DRAWINGS

The foregoing and other aspects and features according to embodiments of the present invention now will be described in detail with reference to the accompanying drawings, in which:

FIGS. 1A-D depict a summary of a phenotypic binning workflow according to the present invention. FIG. 1A Inputs: there are two inputs to the process (1) a set of antibodies known or hypothesized to bind to a target of interest, and (2) a high-content cell-based assay, that captures aspects of the target biology, e.g. activation, alongside other phenotypic behaviors e.g. cytotoxicity; FIG. 1B Antibody testing in assay: In the first step of the process antibodies are synthesized from expression vectors in a suitable expression system in an arrayed format, and added to the high content assay. Next, following a period of incubation the high-content assay is fixed, stained and imaged, or alternatively imaged using live-cell techniques, using an automated microscope, which results in an imaging dataset of the antibodies applied to the high-content assay; FIG. 1C Model training: Prior to training, the dataset is uploaded to a local or cloud image database, that allows programmatic access to the images. Using a weakly supervised approach a deep-neural network is trained on either raw imaging data, or extracted features, to classify the antibody treatment condition that cells/regions of interest belong to. This process allows the extraction of features or embeddings from cells/regions of interest that uniquely define an antibody treatment condition; FIG. 1D Ab binning: Antibodies are clustered into different phenotypic ‘bins’ based on the similarity of their phenotypic effects. This is done through a combination of clustering methods such as K-means or hierarchical optionally coupled with visualization and data-exploration through methods of projecting the embeddings into lower dimensional spaces, e.g. principal component analysis. These results can then be interpreted by experimenters and used to guide follow up studies of antibodies.

FIGS. 2A-D illustrate a workflow for embedding data using a weakly supervised approach. FIG. 2A Region of interest (ROI) identification: either conventional thresholding or convolutional neural networks are used to identify objects e.g. nuclei or cell groups that are hypothesized to be effected by the Ab treatments. ROI's centered on the object are then extracted, the dimensions are selected to capture the object and surrounding environment; FIG. 2B Dataset generation: we create a dataset consisting of single cell crops for every cell in the screen by creating a bounding box around each nucleus (Note; these can also be trained on arbitrarily sized inputs, even entire fields-of-view). The label given to each cell corresponds to the treatment, e.g. antibody or control alongside any other variables e.g. dose. Labels are then encoded as unique one-hot vectors in preparation for network training. Optionally, features from ROI's may be extracted at this point and included, alongside or instead of ROI's, as a training input; FIG. 2C: A deep learning model is trained on the ROI's and/or extracted features. Validation data is used to determine how effectively the network is able to classify ROI's, and once stopping criteria have been reached training is complete; FIG. 2D: As a final step the neural network is shown all cells in the held-out dataset, embeddings are extracted from an intermediate layer—or the final layer in an ‘encoding’ network. In certain circumstances training data and/or validation data might also be included.

FIG. 3A-B provide examples of analysis of embeddings. FIG. 3A: Techniques can be used to project embeddings into lower dimensional spaces for visualization, for example here UMAP is used to transform 64 dimensional embeddings into 2 dimensions, highlighting that addition of TGF-beta cytokine results in phenotypes that diverge from other cytokines; FIG. 3B: Clustering can be performed directly on embeddings to identify phenotypically similar groups and outliers.

FIG. 4A-B detail the application of phenotypic binning to antibody binders of a growth factor receptor shows that a weakly supervised approach replicates traditional supervised analysis when no new phenotypes are present. FIG. 4A: 6 antibodies known to bind to a specific growth factor receptor were tested in a high content assay. Quantification of the results using a 1 dimensional supervised deep-learning approach (supervised MIL) to determine how similar effects are to positive and negative controls identified that three antibodies (Abs 1,2 and 7), that demonstrate inhibition of the growth factor receptor as measured by the high-content assay; FIG. 4B: A weakly supervised analysis, visualized with U-map, highlights that four different phenotypic clusters are present: i) The negative control clusters alongside Abs 5, 6, and 7; ii) Abs 1 and 7 have exactly the same phenotypic effect, and demonstrate a phenotype that differs from the negative control in the direction of positive controls; iii) Ab 2 is closer to the positive controls than the negative control, demonstrating a stronger phenotypic effect that differs from Abs 1/7; iv) Positive controls form a tight cluster that is more divergent from negative controls than any other cluster. Overall this analysis suggests that phenotypes fall on the spectrum of effects between positive and negative controls, since clusters ii and iii lie between the two control groups. The analysis suggests further phenotypic characterization of either Ab1 or 7 alongside Ab2 would be most efficient since Ab1 and 7 have an identical phenotypic effect. The choice between Ab 1 and 7 would be motivated by their physiochemical properties.

FIG. 5A-B depict the application of phenotypic binning to antibody binders of a glycan showing that a weakly supervised approach can identify new phenotypic effects that traditional analysis/methods fail to detect. FIG. 5A: A 1-dimensional supervised deep-learning approach (supervised MIL) fails to show that any antibodies are effecting the phenotypic assay; FIG. 5B: A weakly supervised learning approach shows that antibody binders of the glycan induce an entirely new phenotype that is distinct from either the positive (ii) or negative (i) controls; iii) Abs, 18, 19, 22 and 24 show a weak translocation of p-SMAD2/3 into the nucleus; iv) Ab20 shows a strong translocation of p-SMAD2/3 into the nucleus and also shows reduced p-CREB levels, thus forming its own cluster that is significantly different from either positive or negative controls. Based on this analysis follow up phenotypic studies would focus on one antibody out of Abs 18, 19, 22 and 24 alongside Ab20.

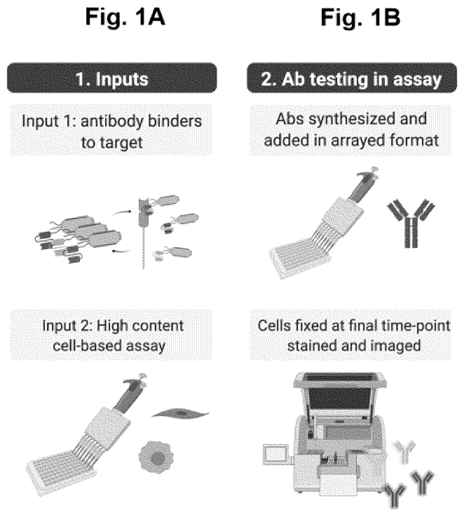

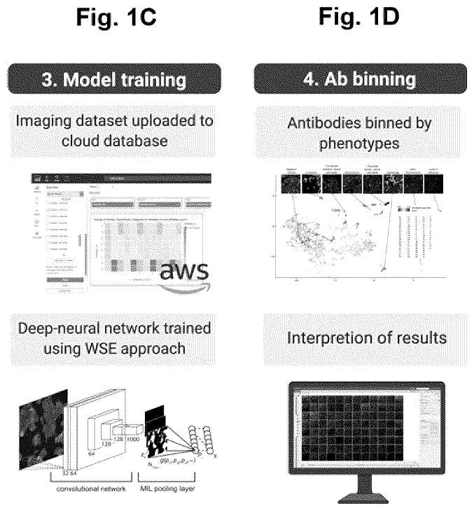

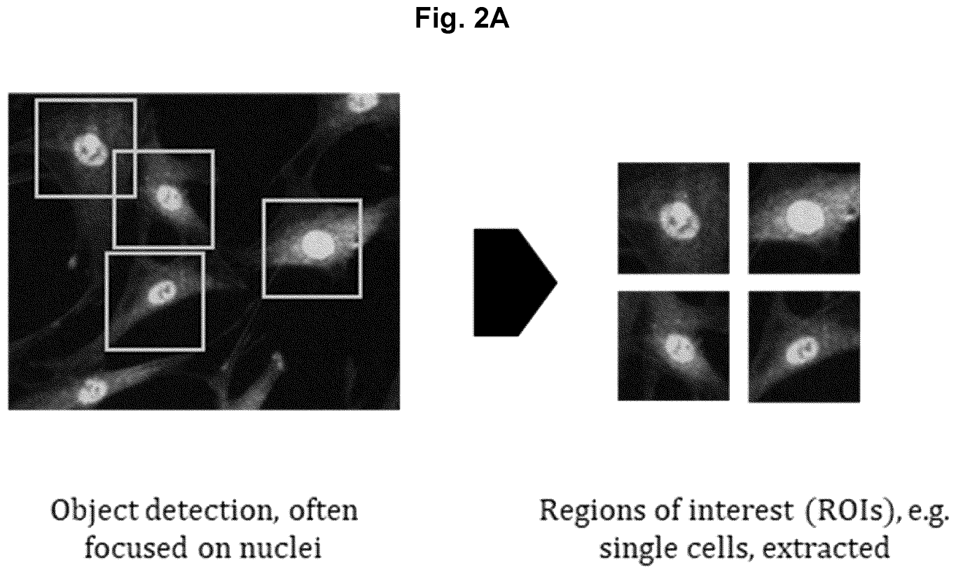

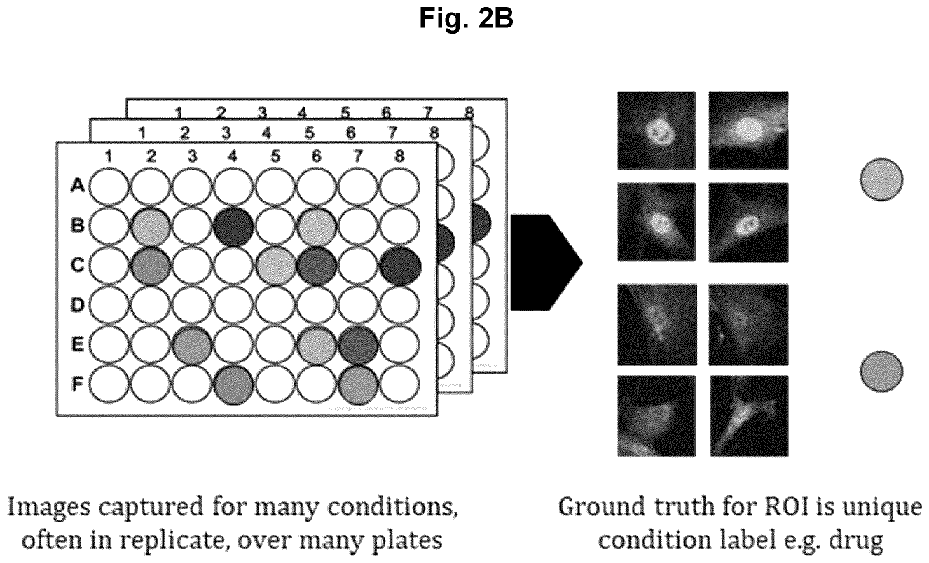

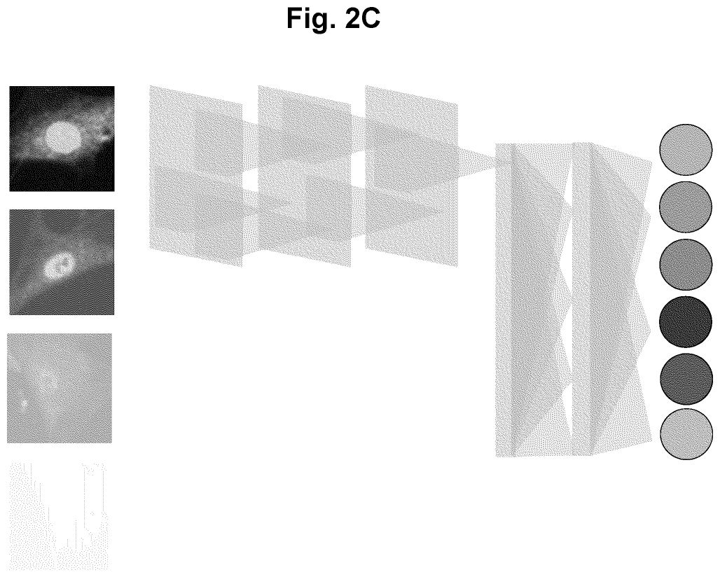

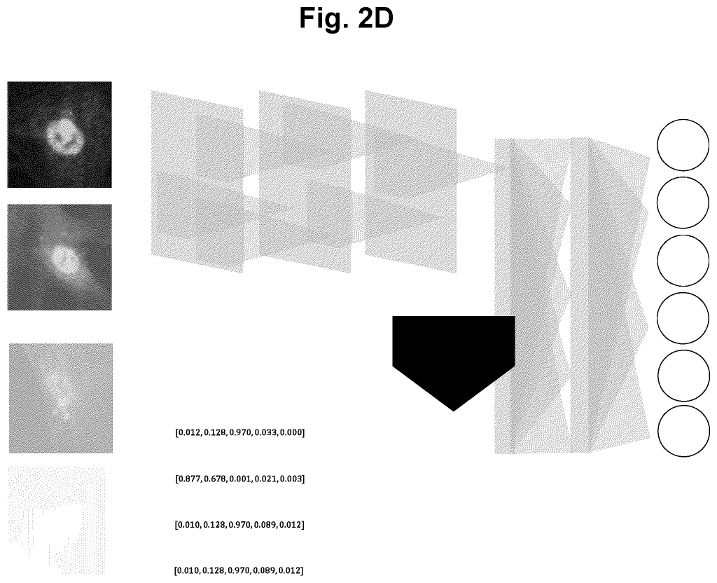

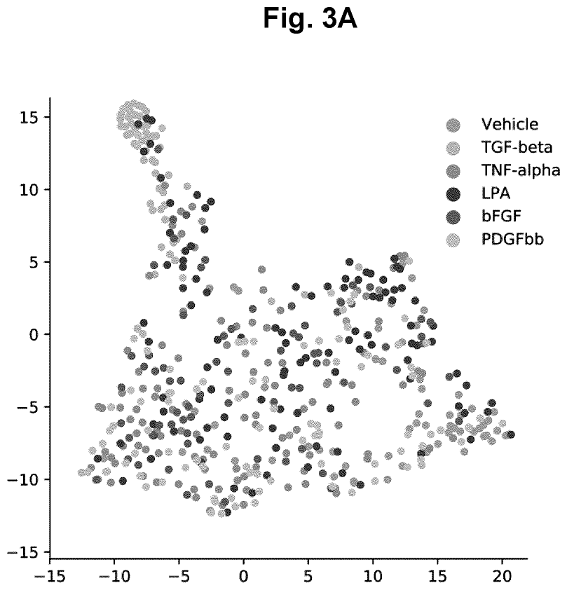

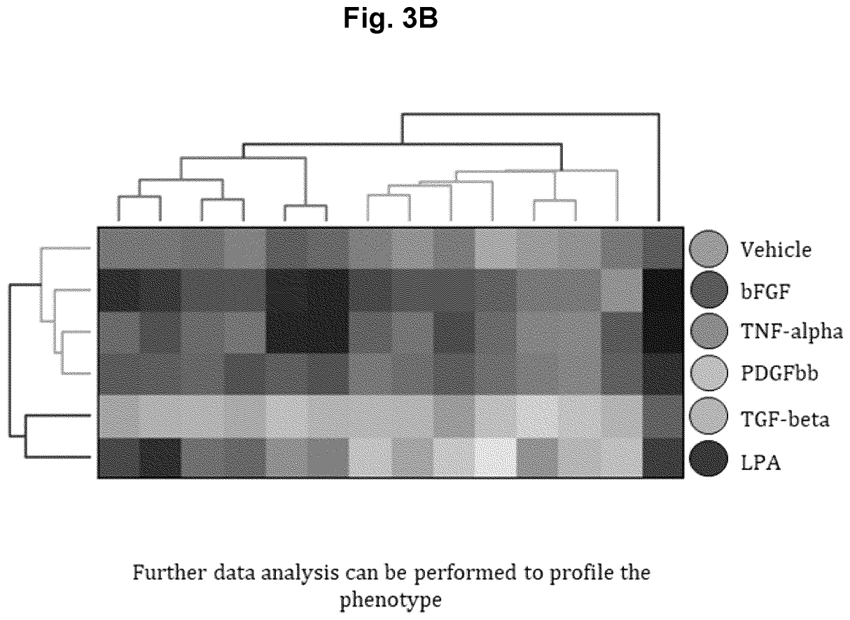

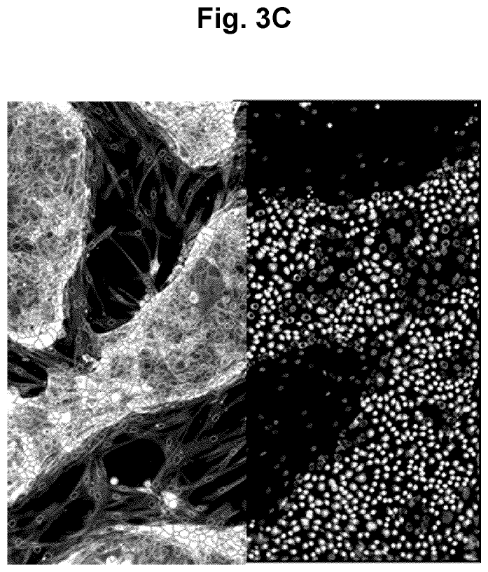

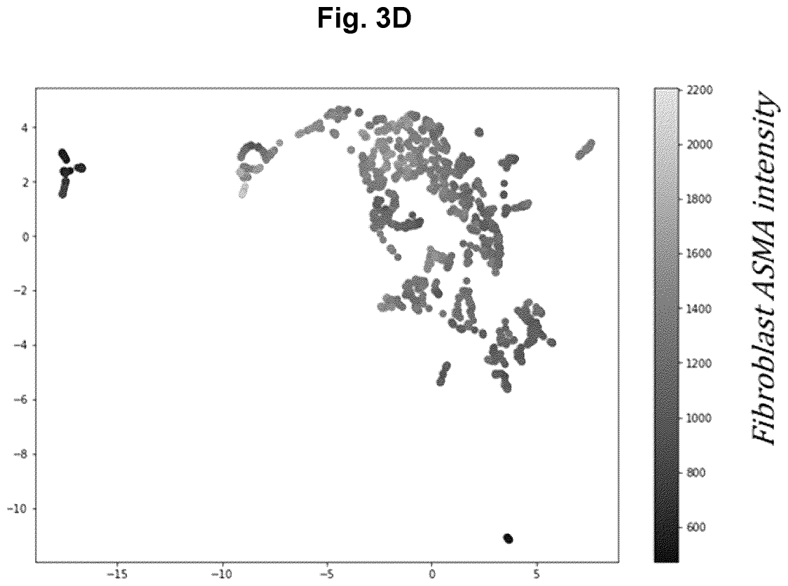

### DETAILED DESCRIPTION

The invention relates to using computer vision and deep neural networks (DNN), including deep convolutional neural networks (CNN) and deep multiple instance learning networks (MIL), to classify cellular phenotypes induced by antibodies. In some aspects, the neural networks use a weakly supervised embedding (WSE) approach.

Described herein are methods and systems for grouping antibodies by their wider effect on a biological systems rather than (or in addition to) the physical location of their binding and their biophysical characteristics. Such phenotypic grouping of antibodies is not a regular feature of discovery pipelines, due to a lack of tools for cost-effectively grouping known and unknown phenotypes together.

Weakly supervised embedding (WSE) techniques have emerged as a strategy for training machine learning models and deep-neural networks to perform a primary task that is auxiliary to what the network is trained to do, such as learning a distance metric between images whilst the model is trained to classify an image. Applied to high-content screening WSE can be used to learn a distance metric between images, through being trained to classify which treatment condition a cell belongs to in a screen. This approach enables training customized models for every screen on large datasets that are typically on the order of tens of gigabytes (GB) to terabytes (TB) or more. WSE can be used to train deep-neural networks on vectors of hand-crafted features extracted from imaging data and/or train deep-convolutional neural networks or deep multiple instance learning networks directly on raw imaging data. Once a model is trained for a screen, features for every image are computed based on intermediate activations in the WSE network, and these features are used for downstream visualization and analysis.

Whilst WSE could be applied to other high-dimensional readouts of cell-based assays such as transcriptomics, high-content screening data is much cheaper to generate and contains key spatial information such as whether two cell-types are near or far from each other and how proteins are organized within an image.

The inventors have determined that this technique enables the cost-effective discovery and grouping of new phenotypes that exist outside of both positive and negative controls. It would be desirable to extend the technique by providing the underlying technology to apply it to identification and grouping of the phenotypic effect of antibodies on arbitrarily complex cell based experimental models.

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine, or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following description provides a practical application and novel hardware arrangement, enabling more efficient classification of antibodies that bind to a protein target. The machine learning techniques described herein, in conjunction with the inputs that the disclosed machine learning system receives, enable assignment of weights to various nodes in various layers within the machine learning system, so as to provide significant enhancement to the efficiency of the overall classification and data analysis process, in a way that human interaction with the raw data could not.

WSE as applied to microscopy datasets employs the training of a neural network to classify images based on metadata defined in the experimental set-up. The auxiliary use of this network is to extract a set of summary features, termed embeddings, describing an experimental condition, field of view, or object (such as a single cell) within a high-content screen relative to other images in the screen, thus the full set of embeddings define a space in which the distance between different phenotypic embeddings is comparable to how similar two phenotypic effects are to one another.

If we define X as the distribution of images (ex: images of single cells) coming from an experiment. Over this distribution of images, we also have a categorical distribution of integer labels C representing the biological condition (ex: antibody treatment conditions). Assume that we have trained a neural network, h:X→C to predict the most likely label c ∈C associated with a given x ∈X. Neural networks consist of multiple layers of computation. This allows us to write h as a composition of two maps that define a new intermediate space. In other words, we can write h=g °f where f:X→E,g:E→C and E is the intermediate vector space with dimensionality defined by the structure of the neural network. We choose an architecture and decomposition of h such that this intermediate space is smaller in dimension than the original input space X. An intermediate representation e=f(x) we refer to as an embedding, and can be thought of as a lower-dimensional feature vector representing the image x.A neural network learns its own intermediate semantic representations to solve the task that it is trained upon. We have empirically validated that, by training a neural network h on a diverse enough array of experimental conditions, the space E can be used to capture the similarity between effects of treatments. More precisely, the learning of h (and consequentially the learning of the map f: X→E) induces a metric Z: E₁×E₂→ [symbol] on the space E which quantifies treatment similarity. For example, suppose we have three embeddings e₁, e₂, e₃ ∈E where the effects of treatments c₁ and c₂ on phenotypes was minimal, and c₃ caused cell death. Then, we would have that Z(e₁, e₂)<<Z(e₁, e₃) and also Z(e₁, e₂)<<Z(e₂, e₃).

In one aspect WSE can be viewed as either as training a single neural network and extracting outputs from intermediate layers, or training two networks and encoding and decoding network, where embeddings represent the final layer of the encoding network. Ordinarily skilled artisans will appreciate that either architecture achieves the purpose of embedding images.

In one aspect, WSE may also include some or all of the following processing steps:

Image normalization and preprocessing—Often images captured from automated microscopes contain technical artifacts that distort future results. Techniques are generally employed to standardize the set of images over an imaging dataset, that may include; 1) Intensity normalization/histogram equalization—pixel values from images in each channel are scaled between a pre-set range such as 0 and 1. This scaling process is susceptible to outliers i.e. exceptionally high-values that distort scaling. Histogram equalization seeks to resolve these issues by aligning the pixel intensity histograms of different images to each other; 2) Background/illumination correction—across a field of view captured by a microscope the average background pixel intensity values can vary between different regions of the field of view, due to variations in illumination intensity in the microscope. To solve this intensity values can be averaged across a large imaging dataset (>100's) of fields of view and this averaged background image negated from each image in the dataset. A second option is to blur fields of view with very wide-angle Gaussian blur functions so as to distort the presence of individual objects, this image can then be negated from individual fields of view.

Object detection—Regions of interest within the microscopy image that include objects hypothesized to vary between conditions must be determined to train the neural network on. Conventional techniques such as Li thresholding and watershed enable detection of high pixel-intensity objects that may, for example, include; 1) Cell nuclei marked using a 4′,6-diamidino-2-phenylindole (DAPI) stain, or similar nuclear marker; 2) multi-cellular organoids detected by probes for cytoplasm, such as phalloidin labelling of F-actin; 3) sub-cellular structures such as the endoplasmic reticulum labelled with concanavalin A. More modern methods for object detection may involve the use of neural networks as well, such as recurrent neural networks, or real-time object detection network. FIG. B-1 depicts the identification of objects within a microscopy image.

Feature Extraction—Images of objects can be processed to extract ‘engineered’ features that may include; 1) Intensity measurements—these capture statistics describing the pixel intensity values seen in a region of interest or object, such as average intensity, standard deviation of intensity, and other higher order metrics; 2) Texture measurements—these look at patterns of pixel intensity values, such as the presence of spots or edges, and are typically measured by performing a 2D convolution of the region of interest or object with a pre-set kernel designed for detection of spots/edges, and then taking the sum or average value of the convoluted image; 3) Morphological measurements—where objects are defined by a 2D region that maps the shape of the objects, properties of the shape can be valuable such as area, length of the longest straight path within the region, and length of the regions perimeter. Extracted features are typically normalized prior to further analysis, such that all features have a standard normal distribution, i.e. the set of feature values has zero-mean and unit standard deviation.

It should be noted that the datasets also may be trained on arbitrarily sized inputs, and even on entire fields-of-view, as is typically the case for multiple instance learning (MIL). The labels associated with each cell are based on metadata extracted from experimental conditions such as drug treatment, concentration, cell line, and fluorescent probe set, as FIG. B-2 depicts. Each unique combination of these values may receive a specific label value. The datasets are stored as TensorFlow Record (TFRecord) files. The TFRecord format is a format for storing sequences of binary records. TensorFlow, an open source machine learning library from Google, can handle this format. The resulting training sets are very large, taking up hundreds of GB. The training data and held-out data may be crops from each condition, or may be technical replicates of a condition FIG. B-2.

These datasets are used to train networks, which may include deep learning models, such as deep-neural networks (DNNs) and convolutional neural networks (CNNs). Where raw imaging data, i.e. regions of interest or whole fields of view are the input it is more typical for a CNN or variant on a CNN to be used for the embedding network that is used in the weakly supervised training procedure. Where extracted features are used, and for the decoding network, it is more typical for a canonical or ‘vanilla’ DNN to be used for the training procedure.

In one aspect, the models may use the residual network (ResNet) architecture of CNN, as a way of training against every unique experimental condition, which is one of the goals. FIG. B-3 depicts one approach to training a deep neural network against various labels, for example, different treatments. Caicedo et al., referenced above, disclose additional techniques, including recurrent neural networks (RNNs), gated recurrent units (GRUs), and Convex combinations of samples. In one aspect these techniques may be employed. However, using the ResNet architecture, such additional techniques may not be necessary.

In another aspect, the following provides a system and method for classifying microscopy images using supervised deep-learning approaches that typically include global pooling, such as, deep multiple instance learning (MIL), and can operate without the prior need of object detection. This method described herein allows the provided system to learn classifiers for full resolution microscopy images without ever having to detect single cells or objects. The system then comprises a convolutional neural network (CNN), typically having an output linked to a pooling layer that is trained to classify negative and positive controls, of which there may be multiple. The CNN comprises an input layer that takes as input a set of microscopy images potentially depicting one or more cell classes and exhibiting cellular densities and florescence related to protein localization, one or more hidden layers for processing the images, and an output layer that produces feature maps for every output category (i.e., cell class) to mimic a specific cell phenotype and/or localized protein. In embodiments, the CNN may provide other outputs in addition to feature maps. An MIL pooling layer that uses representative functions for mapping the instance space to the bag space including, Noising-AND Noisy-OR, log-sum-exponential (LSE), generalized mean (GM) and the integrated segmentation and recognition (ISR) model is applied to the feature maps to generate predictions of the cell classes present in the image. This enables the network to be robust to outliers and large numbers of instances. The result of this step is a neural network that has been trained to separate entire fields of view.

After training, features may be computed for every cell in the dataset. In one aspect, activations from a convolutional layer of the network may be used. In CNN embodiments having multiple convolutional layers, one of those convolutional layers may be used. In one of those embodiments, the fifth convolutional layer may be used. FIG. B-4 depicts the passage of each cell through the neural network, and extraction of numerical features from activations in intermediate layers.

These features that are extracted from the neural network are then used to interpret the phenotypic grouping of images, this occurs through any or all of; 1) Visualization of lower dimensional sub-spaces FIG. C-1; 2) Clustering and analysis of clustering results FIG. C-2; 3) Correlation and analysis with extracted features FIG. C-3. In more detail these methods are:

Visualization—A 2D or 3D visualization of every cell or region of interested in the dataset may be generated by reducing the computed features to two- or three-dimensions using dimensionality reduction techniques such as Uniform Manifold Approximation and Projection (UMAP) or t-distributed stochastic neighbor embedding (tSNE). The resulting points are then plotted in a scatter plot. Interactive plots can also be used to facilitate interactive exploration of the data.

Averaging may be performed over a number of cells or regions of interest in the dataset in order to reduce the level of variation seen between different embedding vectors. The result of averaging is typically clusters that are tighter and better separated. The benefit of this is a visualization in phenotypic clusters can more easily be distinguished with reduced overlap between two similar clusters. The risk is that clusters which are similar to one another become falsely separated, i.e. overfitting.

Clustering—there may be a depiction via clustering. Clustering algorithms may include K-means, K-nearest neighbor, Gaussian mixture models, affinity propagation, and hierarchical clustering, among others. The groupings that these clusters produce identify which experimental conditions induce similar cellular phenotypes. For example, there may be an indication of drugs with similar mechanisms of action.

Comparison to extracted features—Engineered features, such as intensity and morphology measurements can provide a key level of interpretability. By overlaying values of engineered features, either averaged across a condition, or for a specific region of interest, on clusters or visualizations an understanding of what regions of an embedding space corresponds to can be gained. Moreover, statistical measures of how embeddings relate to engineered features, such as the level of correlation of a set of embedding with a feature across the set of conditions can help with interpretation of phenotypic clusters.

In another aspect, the training/validation/test workflow may be performed using data from within the screen being analyzed.

In another aspect, the model also could be transferred from another screen where it has already been trained.

Looking more closely at the antibody profiling method according to aspects of the invention, the method involves the following inputs.

1) A multi-dimensional high-content assay, in which a biological process is modelled in vitro. Using immunofluorescent probes or stains, the in vitro model is imaged. Probes are typically used to identify cell nuclei and cytoplasm to capture morphology. Other probes may be used to model specific protein markers. Probes may be split across multiple replicates of the assay to further increase the dimensionality of the readout, the assay can also be fixed at different timepoints to optimize readouts from different probes. In this fashion, the captured images are combined computationally while the DNNs are being trained. Assays with multiple cell types in each experimental well, complex structures such as organoids or spheroids, and arrayed tissue samples, can also serve as inputs to the process.

2) A library of antibody sequences for testing in the high-content assays. Examples of antibody formats which may be used as sequences can include antigen-binding fragments (Fabs), single-chain variable fragments (scFvs), monoclonal antibodies (mAbs), multi-specific antibodies, and other protein constructs containing regions identified through nucleotide sequence alignment to immunoglobulin genes as being complementarity-determining regions (CDRs), the hypervariable loops responsible for the binding abilities of an antibody.

The process proceeds in steps, as follows.

First, multiple unique variable heavy and light chain sequences may be synthesized as DNA oligomers, or isolated from existing DNA vectors, and ligated into a common heavy and light chain scaffolds, that are part of the same or independent expression vectors. Each expression vector is then transformed or transfected into an expression system to produce antibody protein. One example of such an expression system may be Chinese hamster ovary (CHO) cells while another may be HEK293T human kidney cells. Ordinarily skilled artisans will appreciate that other such expression systems may be suitable. This transformation/transfection is done in an arrayed format such that each antibody is produced in isolation. If independent vectors are used for heavy and light chains, they can be tested in combination. Media containing antibody (conditioned media), or purified antibody, is then tested in a high-content assay, and the results of perturbation are imaged with automated microscopy alongside negative conditions and, in some cases, positive control conditions.

Second, a neural network or other suitable machine learning system may be provided. In one aspect, the neural network is either pre-trained on other experimental datasets, or trained from scratch on the set of images produced from the imaging assays. One meaning of “trained from scratch” that ordinarily skilled artisans will appreciate is that neural networks may be trained from random initializations for a specific screen to be analyzed, using a set of images from the screen to train, and a set to validate. The neural network then is trained on the high-content imaging dataset of different antibody treatments, using the WSE process. Once a sufficient level of accuracy in predicting which condition an object, region of interest, or sub-sample belongs to is achieved using a training and validation dataset, the trained network is then used to extract embeddings for the rest of the high-content screen. Ordinarily skilled artisans will appreciate that other “trainings from scratch” are possible.

Finally, the embeddings generated through the WSE process are used to detect the set of phenotypes present in the imaging dataset; and the antibodies that induce these phenotypes.

Various types of machine learning architectures may be trained in the weakly supervised learning process, including but not limited to DNN, deep CNN, and MIL systems. In one aspect, an MIL system may be layered with a CNN.

### EXAMPLES

### Example 1: Phenotypic Grouping of Functional Antibodies Against a Growth Factor Receptor is Consistent with Existing/Fully Supervised Methods

In this example antibodies that are known to bind to a growth factor receptor (GFR) are screened for the ability to block the activation of fibroblasts induced by GFR ligand. In this cellular model, fibroblast activation refers to the process in which GFR signaling induces fibroblasts to adopt an ‘activated’ phenotype, indicated by an elongated morphology, actin bundling, or a significant change in expression in any one of a number of fibroblast markers. This example demonstrates that by making use of expression-level, localization, and morphological changes, our method is able to group antibodies as phenotypically modulatory (active) or not. This shows that we can replicate existing approaches in cases where no new phenotypes exist.

Inputs: A High Content Assay for Fibroblast Activation and Set of GFR Binding Antibodies

Multiple probes were screened to determine which provided the strongest ability to detect the transformation of both BJ and CCD-8Lu fibroblasts to an activated phenotype. Growth factor, GF was applied to fibroblasts in serum-starved conditions and cells were fixed at 1 hr or 48 hrs post-treatment. Fixed cells were permeabilized, blocked, and stained with probes of interest prior to imaging on a high-content imaging microscope (GE Incell 6000). Image crops of single cells were embedded into a high dimensional space using a weakly supervised approach. The accuracy of the network in being able to predict whether an image crop of a single cell belonged to a positive exogenously-applied GF condition or negative control condition was recorded for 30 probes at the 1-hr time point and 14 probes at 48-hr time point. Single cell crops of BJ and CCD-8Lu cells were embedded with a weakly supervised network, and the prediction accuracy of the network in distinguishing GF positive and negative crops was calculated on held out data (20%). Each well of treated BJ fibroblasts or CCD-8Lu lung fibroblasts were fixed, permeabilized, and stained with Hoechst-33342 for labelling DNA, Phalloidin for labelling F-actin (allowing for visualization of cell boundaries) and two commercial antibodies targeting specific cellular proteins. We demonstrated, in line with existing literature, that at 1 hour the phosphorylated-Akt1 (p-Akt), phosphorylated-GSK-3beta (p-GSK3b), and phosphorylated-CREB (p-CREB) probes provide the highest classification accuracy for identifying GF stimulation, whilst at 48 hours the induction of paxillin and RhoA are the most accurate readouts of fibroblast activation. Together these experiments identified an optimized combination of probes and GF concentration that serve as the high-content assay input to our antibody discovery process.

The second input to the process was a set of antibodies that are known to bind to a soluble extracellular domain of the GFR. An scFv library of greater than 10¹¹ diversity was applied to biotinylated GFR extracellular domain and selections were performed in both phage display and yeast display formats following established protocols. Regions corresponding to the VL, VH, and full scFv segment were amplified by PCR, and amplicons were purified using a PippinHT. Amplicon sequencing reads were obtained by next-generation sequencing (NGS) at a read depth of 50000 reads per sample. The results of NGS on the VL and VH sequences allowed for them to be ranked by frequency of sequence per round of selection. The results of NGS on the full-length scFv amplicons provided paired reads of 150-200 base pairs from either end of the scFv amplicon, allowing for post-sequencing alignment of complete VL and VH sequences and thereby providing a method for identifying VL-VH pairings, overcoming the 250 base pair read length limitation of Illumina's 2×250 MiSeq NGS technology. When combined, these two NGS read strategies identified 8 VL sequences and 12 VH sequences that increased in frequency during selections and provided probable pairing between those VL and VH sequences.

In the first step of the phenotypic binning method, all 20 identified VL and VH sequences were synthesized and cloned into individual mammalian expression vectors containing the appropriate IgG1 kappa CL, IgG1 lambda CL, or IgG1 CH1-CH2-CH3 sequences. Expression of IgGs was performed by transfecting 293F mammalian expression cells with light chain and heavy chain vectors in a combinatorial manner in 1 mL volume, 96-well format. After seven days of expression, 293F cultures were centrifuged and the resulting media was filtered in 96-well format to remove cells and cellular debris from supernatant. Supernatant was then analyzed for presence of IgG by running samples through a LabChip GXII Touch protein analyzer in both non-reducing and reducing conditions. Sample separation patterns were compared between negative (non-transfected) controls and between reduced and non-reduced samples in order to detect bands corresponding to expressed IgG, in the 140-170 kDa range. IgG supernatant binding to antigen was detected using horseradish peroxidase (HRP) conjugated human Fab″-binding goat IgG.

Assay plates containing monocultured cell lines were prepared as outlined previously, and after 24 hours of starvation were treated with either unpurified IgG supernatant or IgG purified by standard protein-G methods. IgG treatments were applied in the presence of GF to monocultured fibroblasts. Plates were then prepared for imaging as described previously at both 1-hr and 48-hr time points. An automated microscope was used to capture nine fields of view (FOV) per well. Both purified antibodies and conditioned media were tested against both BJ skin fibroblasts and CCD-8Lu lung fibroblasts. Treatments were applied to at least two wells per cell line (24 FOV), while controls were applied to at least 4 wells per cell line (48 FOV). This generated a total of 12 independent tests at the 1 hour timepoint and 12 independent tests at the 48 hour timepoint. Overall a total of 1080 FOVs were captured, this included 144 positive control FOVs, 144 negative control FOVs, and 144 FOVs from a control therapeutic antibody targeting GFR.

As the second step of the process a WSE model was trained on the imaging dataset. Nuclei were detected belonging to single-cells in the image using a traditional nuclei detection approach in which intensity based thresholding is applied to blurred images of nuclei stained with DAPI to generate a binary mask covering areas of high DAPI intensity. Binary erosion and dilation operations were applied to reduce the number of erroneously detected nuclei. The binary mask was used to define a foreground region in the original blurred nuclei image. Within this foreground region, a peak finding algorithm was used to identify and label nuclei centers—those centers that were too close to one another were discarded as belonging to the same nuclei. The result of this process is an image in which the center of each nuclei is labelled by a pixel with a unique integer value. 64×64 pixel crops across four channels (DAPI, FITC, dsRed, and Cy5) were then taken around each nuclei center, to create a set of nuclei crops for every image/experimental condition in the high-content screen.

The set of nuclei crops was divided into train, validation, and test datasets, with each crop labelled according to the condition it was taken from (antibody or +/− control). A CNN architecture, similar to ResNet, consisting of 5 blocks of convolutional layers, with 3 convolutional layers per block, separated by max-pooling layers was trained using the WSE process. Pooling of convolutional layers was performed over a single crop and consisted of concatenating the set of max-pooling layer outputs in the final convolutional block. A single fully connected layer was then applied to this max-pooling layer prior to the layer from which we anticipated extracting embeddings. Across all of these layers a Linear Rectifier function (ReLu) was used to introduce non-linearity into the network. After the embedding layer, a single additional layer was used to generate a prediction of the condition that the 64×64 pixel crop belonged to. The network was trained for 50 epochs (iterations through the full training dataset), until no further improvement in the prediction accuracy achieved on validation data could be seen across 3 epochs. The trained network was then applied to the full set of 64×64pix single-cell crops. For each crop, a 256-dimensional feature vector was extracted—this represented a 16-fold reduction in the dimensionality of the input data.

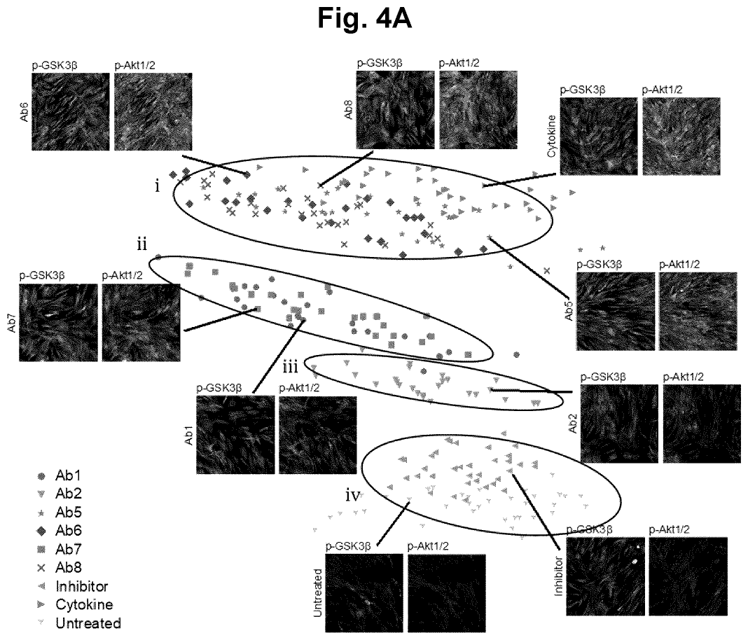

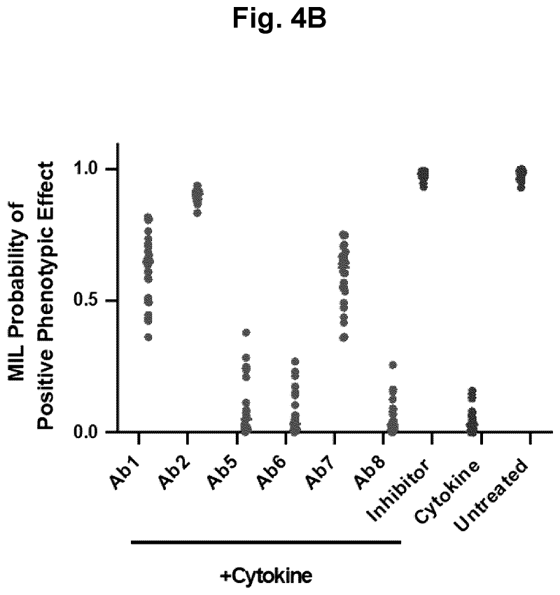

In the final step of the phenotype grouping process, we used UMAP to reduce the dimensionality of the embedding space down to 2 dimensions for visualization FIG. 4A. In the resulting plot clear clusters could be seen corresponding to the negative control (Cytokine added) and positive controls (Untreated and a known growth factor receptor inhibitor). Several antibodies grouped with the negative controls indicating that despite binding the target they had no phenotypic effect. Lying between the positive and negative controls lay two phenotypic clusters; one group lay closer to the negative controls and contained Abs 1 & 7; Ab 2 meanwhile fell closer to positive controls, indicating a stronger inhibitory effect on the target than the other two antibodies.

### Output: Detection of Functional Antibody Inhibitors of the GFR

This set of independent tests revealed that three of the antibodies tested were functional inhibitors of GF signaling, all effects lay directly between positive and negative controls indicating a continuous spectrum of inhibition. Comparing this example with a supervised, one dimensional MIL network trained on the positive and negative control examples FIG. 4B, we find that the two approaches both arrive at the same result that Ab2 is most similar to positive control data, i.e. has the strongest effect, whilst Ab's 1 and 7 have significant but weaker effects. Overall this example highlights the process employed and shows that it can reproduce the results of more straightforward supervised methods when hit's lie directly between positive and negative controls in phenotypic space.

### Example 2: Phenotypic Grouping of Antibodies Targeting a Glycan Results in Discovery of a New Phenotype

In this example, a cell-based model is developed to capture a disease process associated with the activation of fibroblasts, as per Example 1. Here antibodies that bind to a glycan target were identified from a phage display library. Unlike Example 1, antibodies were found that caused phenotypic effects which diverged from what was expected based on positive and negative controls. This example shows the value of our approach in being able to detect new phenotypes that diverge from what is expected prior to the high-content screen.

Similar to the Example 1, as the first input to the process, a high-content assay was developed in which BJ and CCD-18Co fibroblasts were stimulated with cytokines in order to induce a CAF-like phenotype in fibroblast cells. Here, response to TGF-beta cytokine was measured using a variety of reporters at 1 hr and 48 hrs that included pCREB, pERK, and SMAD proteins at 1 hr, as well as aSMA, PDGFRB, Paxillin, and CD248 at 48 hrs.

As the second input, antibodies that selectively bound to the Glycan target were identified as set out in Example 1. In total 8 antibodies were identified through this process, and of these 6 were found to bind the target as measured by ELISA.

Weakly supervised learning was used to profile the set of phenotypes induced by the antibodies. The imaging dataset was segmented on the nuclear channel (DAPI) in order to identify regions of interest that corresponded to single cells as per Example 1. Again a CNN architecture was trained on a subset of the extracted single-cell crops. After 10 epochs the trained network was applied to the full set of single-cell crops and 256-dimensional feature vectors [embeddings] were extracted from the second to last layer.

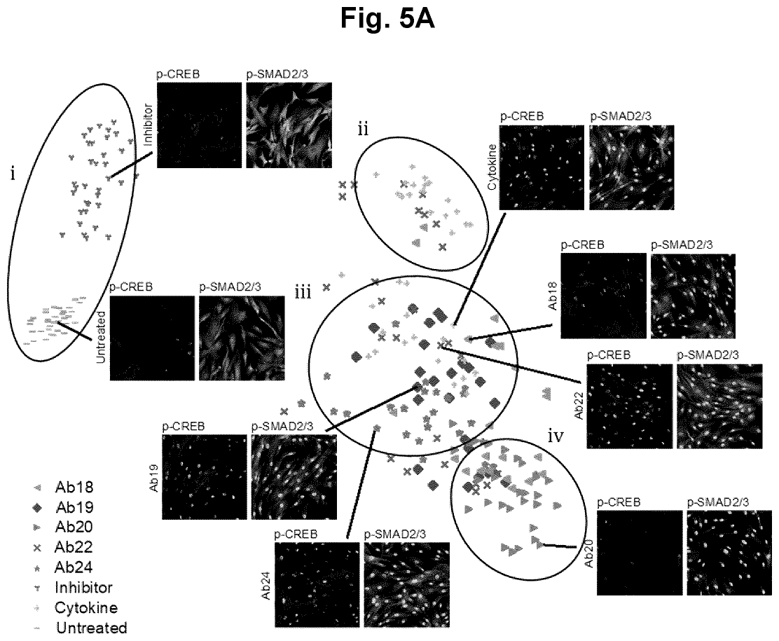

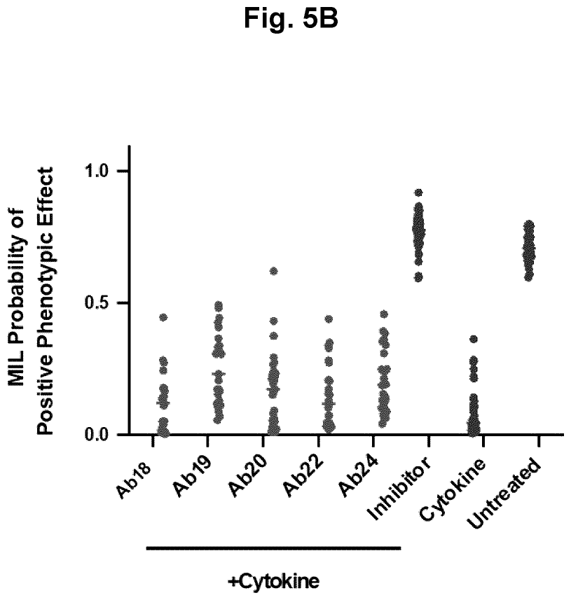

As the final step of the process, to determine the set of phenotypes learnt by the deep-neural network, we reduced the dimensionality of the data using UMAP. Applying UMAP to the averaged embeddings taken from this screen allowed us to detect clear separation between phenotypic clusters FIG. 5A. Specifically, we found that unlike Example 1, where phenotypes clustered into two groups consisting of antibodies with effects similar to either positive and negative controls, a non-control phenotypic effect could be detected that contained antibody binders to the glycan target.

By correlating a set of engineered features with the groupings over many different fluorescent channels, imaged across technical replicates of the experiment, it could be seen that negative controls had nuclear SMAD protein, and low levels of cytoplasmic pCREB. Positive controls had cytoplasmic SMAD and higher levels of cytoplasmic pCREB. In contrast to either of these groups 4 Ab binders of the Glycan induced a phenotype of nuclear SMAD and high levels of Nuclear pCREB. Specifically, Abs, 18, 19, 22 and 24 showed a weak translocation of p-SMAD2/3 into the nucleus. Finally Ab20 formed it's own group demonstrating strong nuclear translocation SMAD and pCREB inhibition similar to negative controls. Overall suggesting that the antibody binders induced two distinct phenotypes in this system.

This demonstrated that these antibodies could have anti-inflammatory properties—since nuclear CREB is marker of production of IL-10 an anti-inflammatory cytokine, without the potentially pro-fibrotic effects associated with TGF-beta signaling and SMAD nuclear translocation. Together this highlights the value in this method for being able to quickly and automatically detect new phenotypes and then in turn relate them back to changes in proteins expression levels, phosphorylation, and/or localization within cells.

### Output: Detection of Functional Antibodies Against a Membrane Glycan and a New Phenotypic Effect

The outputs of the process applied to this set of antibodies and high-content assay was the finding that all 5 antibody binders had an effect that was dissimilar to either the positive or negative controls, highlighting the power of WSE for grouping phenotypes. In contrast, where a more straightforward fully supervised approach is used the phenotypic effects of the antibodies go undetected, resembling positive controls FIG. 5B. As a result of this approach a representative Ab from groups iii and iv would be progressed forward into further phenotypic testing that might otherwise have been missed. Overall, this example highlights how a weakly supervised learning process can be used to group the phenotypic effects of antibodies with no prior knowledge of what the phenotype is, and independently of example control phenotypes. Providing novel insights and antibody hits that might be missed with conventional methods.

While numerous embodiments in accordance with different aspects of the invention have been described in detail, various modifications within the scope and spirit of the invention will be apparent to ordinarily skilled artisans. In particular, certain methods are disclosed, as well as individual steps for performing those methods. It should be understood that the invention is not limited to any particular disclosed sequence of method steps. Consequently, the invention is to be construed as limited only by the scope of the following claims.

What is claimed is:

1. A method for profiling antibodies, comprising:
providing a machine learning model receiving one or more inputs taken from an image selected from one of a plurality of images generated from one or more groups of imaging assays, wherein the image is associated with other images of the one or more groups according to one or more antibodies present in a biological sample from which the image was generated; and
using the machine learning model, computing an output comprising one or more predicted phenotypes represented in one or more of the plurality of images of that group, the output comprising an antibody profile.

2. The method according to claim 1 wherein said biological sample comprises a plurality of cells.

3. The method according to claim 2 wherein said plurality of cells comprises two or more different cell types.

4. The method according to any one of claims 1-3 wherein the machine learning process is weakly supervised, and the model is neural network selected from the group consisting of a multiple instance learning (MIL) model, a deep neural network (DNN), a convolutional neural network (CNN), and a neural network comprising one or more convolutional layers and an MIL pooling layer.

5. The method according to claim 1 further comprising training the machine learning model using the plurality of images, to enable the model to predict a group from the one or more groups of which the image is a member.

6. The method according to claim 5, wherein the one or more groups come from a common experimental design with other images.

7. The method according to claim 5, wherein the training further comprises enabling the model to predict whether an image from a group was generated from one or a plurality of experimental conditions in the assays from which the images were generated.

8. The method according to claim 7, wherein the experimental conditions are selected from the set of antibody identifier, concentration of antibody, cell type, combination of cell types, cell seeding density, probe set, presence, absence or concentration of ligand, presence, absence or concentration of small molecule inhibitors, depletion, knockout, overexpression or modulation of genes, or presence, absence or concentration of combinations of any other molecules that may modulate the activity of an antibody.

9. The method according to claim 7, further comprising providing a graphical display of phenotype versus antibody class grouped according to a distribution of learned weights in one or more hidden layers of the trained machine learning model.

10. The method according to claim 1, wherein the inputs comprise pixel intensities.

11. The method according to claim 10, wherein said pixel intensities correspond to a plurality of fluorescent probes in said biological sample in said assay, each probe representing a phenotype of interest.

12. The method according to claim 11, wherein at least one probe represents an on-target phenotype and at least one probe represents an off-target phenotype.

13. The method according to claim 12, wherein said on-target phenotype is cell stimulation and/or expansion.

14. The method according to claim 12, wherein said on-target phenotype is cell senescence, apoptosis and/or cytotoxicity.

15. The method according to claim 12, wherein said on-target phenotype is stimulation of a cell-signaling pathway.

16. The method according to claim 12, wherein said on-target phenotype is inhibition of a cell-signaling pathway.

17. The method of claim 1 wherein the output comprises a graphical representation of the antibody profile.

18. The method according to any one of the preceding claims, wherein the output antibody profile comprises a predicted classification of phenotype, wherein the phenotype is selected from the group consisting of on-target and off-target.

19. The method of claim 18, wherein the on-target phenotype comprises inhibition of an activated state of a tumor cell, a non-tumor cell, or a cell in contact with a tumor cell.

20. The method of claim 19, wherein the on-target phenotype comprises inhibition of activation of a non-tumor cell fibroblast by an exogenously applied activating ligand.

21. The method of claim 19, wherein the on-target phenotype comprises inhibition of an activated state of a cell in contact with a tumor cell.

22. The method of claim 21, wherein the cell in contact with the tumor cell is a fibroblast.

23. The method of claim 22, wherein the on-target phenotype comprises inhibition of tumor cell contact-induced fibroblast activation.

24. The method of any one of claims 18 to 23, wherein the off-target phenotype is selected from the group consisting of autophagy, cytotoxicity, auto-fluorescence, and senescence induction.

25. The method according to claim 1, further comprising a preliminary step of individually contacting each of a panel of antibodies with a biological sample, wherein said sample comprises a probe set representative of a plurality of phenotypes of interest, and generating at least one image of each antibody/biological sample pairing in said panel.

26. The method according to claim 25, comprising generating two or more images of each antibody/biological sample pairing at sequential time points.

27. The method according to claim 25, wherein at least one probe represents an on-target phenotype and at least one probe represents an off-target phenotype.

28. A method for profiling antibodies based on phenotypic effect and/or activity, comprising the steps of:
a) contacting a plurality of antibodies [same target only? multiple targets? Quantify and characterize antibodies in dependent claims] with a biological sample in an arrayed format, wherein said biological sample comprises one or more cell types comprising a plurality of labeled probes to create a high-content assay;
b) imaging said high content assay with automated microscopy to generate an imaging dataset;
c) applying a deep neural network to said imaging dataset to detect the set of phenotypes present in the imaging dataset; and determine the antibodies that induce one or more of these phenotypes.

29. The method according to claim 28, wherein weakly supervised embedding is used to train the deep neural network on the phenotypic similarity between different images.

30. The method according to claim 29, wherein the deep neural network is trained on a plurality of extracted features encompassing variations between regions of interest in each image in the dataset to embed the imaging dataset.

31. The method according to claim 30, wherein regions of interest comprising extracted features that are unique to an experimental condition are passed into the deep neural network and the result of the prediction on a subset of training data is used to directly update the weights in the deep neural network.

32. The method according to claim 31, wherein an unsupervised clustering technique is used to identify discrete phenotypic groups defined by a threshold level of similarity between extracted features.

33. A system, comprising
a data repository of imaging assays for a library of antibodies against one or more high-content cell-based assays;
one or more processors coupled to the repository; and
machine executable code, residing in non-transitory memory accessible to the one or more processors, that when executed by the one or more processors performs the method of claim 1.

34. A computer-implemented machine learning architecture for profiling antibody activity in a high-content assay, the machine learning architecture executed on one or more processing units, the machine learning architecture comprising:
a machine learning model receiving one or more inputs taken from an image selected from one of a plurality of images generated from one or more groups of imaging assays, wherein the image is associated with other images of the one or more groups according to one or more experimental conditions including antibody treatments present in a biological sample from which the image was generated;
the machine learning model comprising an input layer receiving the one or more inputs, and one or more hidden layers of processing nodes, each processing node comprising a processor configured to apply an activation function and a weight to inputs of the processor, a first of the hidden layers receiving an output of the input layer and each subsequent hidden layer receiving an output of a prior hidden layer; and
at least one of the one or more hidden layers configured to generate one or more class specific predictions for cellular features of one or more cell classes present in the images, wherein the class specific predictions represent probabilities of the cell classes for an image; and
an output layer, responsive to the learned weights from one or more of the hidden layers and the probabilities of the cell classes, to generate an antibody profile according to the weights and probabilities of the cell classes.

35. The computer-implemented machine learning architecture of claim 34 wherein the antibody profile comprises a graphical representation.

36. The computer-implemented machine learning architecture of claim 34 wherein one or more of the hidden layers comprise a convolutional layer.

37. The computer-implemented machine learning architecture of claim 34 wherein the machine learning model is selected from the group consisting of an MIL model, a deep neural network (DNN), a convolutional neural network (CNN), and a neural network comprising one or more layers.
